(19) United States
(12) Patent Application Publication
(10) Pub. No.: US 2004/0205845 A1
(43) Pub. Date: Oct. 14, 2004

(54) Transgenic Plants Showing an Increased Accumulation of Starch

(75) Inventors: Babette Regierer, Berlin (DE); Alisdair R. Fernie, Berlin (DE); Peter Geigenberger, Dossenheim (DE); Jens Kossmann, Roskilde (DK)

Correspondence Address: BIRCH STEWART KOLASCH & BIRCH, PO BOX 747, FALLS CHURCH, VA, 22040-0747, US

(73) Assignee: Max-Planck-Gesellschaft zur Forderung der Wissenschaften e.V., Berlin (DE)

(21) Appl. No.: 10/474,487

(22) Filed: May 7, 2004

(30) Foreign Application Priority Data
Apr. 9, 2001 (EP) EP 01108864.8

(51) Int. Cl. A01H 1/00; C12N 15/82

(52) U.S. Cl. 800/288

(57) ABSTRACT

### Abstract of the Disclosure

Described are transgenic plants showing an increased accumulation of starch and/or an increased yield in starch-storing parts, organs or tissues due to a reduction of the endogenous adenylate kinase (ADK) activity in cells of the plant. Accordingly, such a reduction can be achieved by introducing a nucleic acid molecule, e.g. a nucleic acid molecule encoding a suitable antisense RNA, into the plant genome. Furthermore, recombinant nucleic acid molecules and methods for the production of such plants are described.

### Detailed Description of the Invention

### Cross Reference To Related Applications

This application is the national phase under 35 U.S.C. § 371 of PCT International Application No. PCT/EP02/03962 which has an International filing date of April 9, 2002, which designated the United States of America.

### Brief Description of Drawings

Figure 1shows a map of the vector pBinAR-Kan containing the cDNA sequence of the potato plastidial adenylate kinase (StpADK) cloned between the constitutive CaMV 355 promoter (Franck, Cell 21 (1980), 285-294) and the A. tumefaciens octopine synthase gene terminator (ocs) in antisense direction.

Figure 2shows a Northern blot of StpADK transcript level in leaves from wild type and transgenic lines. The probe was made from full length cDNA encoding StpADK by the riboprobe method.

Figure 3shows the adenylate content of transgenic lines. The data is presented as the mean SE of six individual plants per line.

Figure 4shows the starch content of transgenic lines. Starch was determined in the same samples from developing tubers used for the analysis of adenylates presented in Figure 3. The data is presented as the mean SE of six individual plants per line.

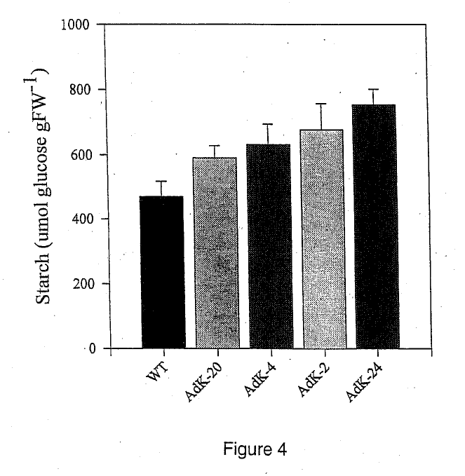

### Detailed Description

The present invention relates to transgenic plants showing an increased accumulation of starch and/or an increased yield in starch-storing parts, organs or tissues due to a reduction of the endogenous adenylate kinase (ADK) activity. According to the present invention, such a reduction can be achieved by introducing a foreign nucleic acid molecule, e.g. a nucleic acid molecule encoding a suitable antisense-RNA, into the plant genome. Furthermore, the present invention relates to recombinant nucleic acid molecules and to methods for producing the disclosed plants.

There has been considerable interest to increase starch accumulation within starch storing tissues of plants, e.g., in potato tubers, either by conventional plant breeding or genetic manipulation strategies (Schafer-Pregl et al., Genetics 97 (1998), 834-846; Stark et al., Science 258 (1991), 287-292; Trethewey et al., Plant J. 15 (1998), 109-118). Transgenic approaches have focused primarily on the modulation of either sucrose catabolism (Sonnewald et al., Nature Biotech. 15 (1997), 794-797; Trethewey et al., loc. cit.) or of the plastidial starch synthetic pathway (Stark et al., loc. cit.; Tauberger et al., Plant J. 23 (2000), 43-53). To date the only successful transgenic approaches have resulted from the overexpression of either a bacterial AGPase (Stark et al., loc. cit.) or of the Arabidopsis amyloplastidial ATP/ADP translocator (Tjaden et al., Plant J. 16 (1998), 531-540). Attempts were previously made to improve the starch yield of potato tubers by the expression of a more efficient pathway of sucrose degradation, consisting of a yeast invertase and a bacterial glucokinase (Trethewey et al., loc. cit.). However, despite the fact that the transgenic tubers exhibited reduced levels of sucrose and elevated levels of hexose-phosphates, ATP and 3-PGA with respect to wild type tubers this attempt failed. This is intriguing, since hexose phosphates, ATP and 3-PGA represent both the immediate precursors for and the activator of AGPase, respectively (for review see Preiss, In: The Biochemistry of Plants, Vol. 14, Academic Press, San Diego, California (1988), 181-254). Moreover, these hexose-phosphates were found to partition to glycolysis at the cost of starch synthesis resulting in a decreased starch accumulation within these lines (Trethewey et al., loc. cit.). When taken together all these studies reveal that the regulation of starch synthesis, e.g., in potato tubers is more complex than initially expected and a simple increase in the concentration of the precursors may not be sufficient to drive starch synthesis. Thus, there is still a need for approaches which allow to effectively increase the starch accumulation in starch storing tissues of plants.

Therefore, the technical problem underlying the present invention is the provision of plants showing an increased starch accumulation as well as of means and methods for their production.

This technical problem is solved by the provision of the embodiments as characterized in the claims.

Accordingly, the present invention relates to a transgenic plant the endogenous adenylate kinase (ADK) activity of which is reduced.

It has been surprisingly found that the repression of plastid-localized adenylate kinase in potato tubers significantly increases the accumulation of starch in the tubers. Further improvements that result from this ADK repression comprise increases of the tuber yield (of up to 184%), of the density of the tubers, of the adenylate content and of the amino acid content, in particular of essential amino acids. From Tjaden (Plant J. 16 (1998), 531-540), it has been known that an elevated ATP content in the plastids leads to an increase in starch accumulation. This was achieved by overexpressing the plastidial ATP/ADP-translocator. However, that the repression of the plastidial ADK would have a similar effect on the adenylate content in these organelles was not expected. The enzyme adenylate kinase (EC 2.7.4.3) catalyses the following reaction:

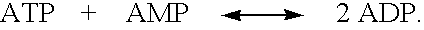

The ADK the activity of which is reduced according to the provisions of the present invention may have any possible subcellular localization. Cytoplasmic ADK isoenzymes have for example been described by Moore (Plant Science Letters 35 (1984), 127-138). The nucleotide sequence of two cytoplasmic ADK isoenzymes from rice was described by Kawai (Plant J. 2 (1992), 845-854 and Plant Mol. Biol. 27 (1995), 943-951). ADK isoenzymes with a mitochondrial localization may likewise be used in the embodiments of the present invention. Preferred, however, are ADKs having a plastidial localization since the plastids, in particular the amyloplasts, are the sites of starch biosynthesis in plant cells. Suitable plastidial ADKs are described in the literature as for example that of () maize (Schiltz, Eur. J. Biochem. 222 (1994), 949-954). Furthermore published are expressed sequence tags (ESTs) exhibiting a pronounced homology to the above ADK-encoding nucleotide sequences namely one from Glycine max (GenBank EMBL database accession no. BE 022879), two from Arabidopsis thaliana (accession no. N37538) and one from the swollen stolon of potato (accession no. AW 905934). In a preferred embodiment, the above-mentioned ADK to be reduced in activity is encoded by a polynucleotide selected from the group consisting of:(a) polynucleotides encoding a polypeptide having the amino acid sequence depicted in SEQ ID NO: 2;(b) polynucleotides comprising the nucleotide sequence depicted in SEQ ID NO: 1;(c) polynucleotides hybridizing to the complementary strand of the polynucleotide of (a) or (b); and(d) polynucleotides the nucleotide sequence of which deviates from the nucleotide sequence of a polynucleotide of (c) due to the degeneracy of the genetic code.

The cDNA encoding the plastidial ADK of potato (StpADK) having the nucleotide sequence as shown in SEQ ID NO: 1 was cloned as described in the appended Examples in order to be used to prepare transgenic antisense potato plants. The term "hybridizing" refers in this context to hybridization under conventional hybridization conditions, preferably under stringent conditions, as for instance described in Sambrook at al., Molecular Cloning, A Laboratory Manual, 2^{nd} edition (1989) Cold Spring Harbor Laboratory Press, Cold Spring Harbor, NY. In an especially preferred embodiment the term "hybridizing" means that hybridization occurs under the following conditions:

| | |
|---|---|
| Hybridization buffer: | 2 × SSC; 10 × Denhardt solution (Fikoll |
| | 400 + PEG + BSA; ratio 1:1:1); 0.1% |
| | SDS; 5 mM EDTA; 50 mM Na_{2HPO}_{4;} |
| | 250 μg/ml of herring sperm DNA; |
| | 50 μg/ml of tRNA; or |
| | 0.25 M of sodium phosphate buffer, |
| | pH 7.2; 1 mM EDTA |
| | 7% SDS |
| Hybridization temperature T = | 60° C. |
| Washing buffer: | 2 × SSC; 0.1% SDS |
| Washing temperature T = | 60° C. |

Advantageously, polynucleotides encoding an ADK have a nucleotide sequence of at least 90%, preferably of at least 95%, more preferably of at least 98% and most preferably of at least a 99% identity to the nucleotide sequence of SEQ ID NO:1. Likewise, such polynucleotides encode a polypeptide having an amino acid sequence of at least 80%, preferably of at least 90%, more preferably of at least 95%, still more preferably of at least 98% and most preferably of at least a 99% identity to the amino acid sequence of SEQ ID NO:2.

The term "endogenous adenylate kinase (ADK) activity" refers to any enzymatic activity in a plant cell that catalyses the above-shown reaction. Preferably, said activity is localized to the plastids, such as, e.g., chloroplasts and, in particular, amyloplasts, and may therefore preferably be measured by using isolated pastids (see, e.g. Haake (Plant J.14 (1998), 147-157 for a suitable method for plastid isolation). ADK activity can be determined by methods described in the literature as for example in Kleczkowski (Plant Physiol. 81 (1986), 1110-1114). The term "endogenous" refers to ADK activities that are present in the source plants, advantageously wild-type plants, which are taken as starting point for applying the herein described embodiments. By a "reduction of ADK activity" as referred to above is meant a reduction by at least 20%, preferably 25% and most preferably 30% compared to the corresponding endogenous ADK activity in the source plant. Likewise, a reduction of the endogenous ADK activity may be determined by measuring the amount of the corresponding ADK transcript or protein in the plant cell. Thereby, a reduction of the ADK transcript by at least 30%, preferably by at least 50 to 70% and most preferably by at least 70 to 90% compared to the corresponding amount of transcript in the source plant is indicative for a transgenic plant according to the present invention. On the protein level, a reduction by at least 30%, preferably by at least 50 to 70% and most preferred by at least 70 to 90% of the ADK polypeptide compared to the corresponding source plant provides for an efficient increase of starch accumulation in the transgenic plants of the invention.

The term "increased accumulation" in this context means that the transgenic plants according to the invention have an increased starch content when compared to corresponding non-transformed wild-type plants. Preferably, the starch content in starch storing parts, organs or tissues of the plant is increased. In this context the term "increased" means an increase in starch content of at least 10%, preferably of at least 20%, more preferably of at least 40%, even more preferably of at least 60% and particularly preferred of at least 80% when compared to corresponding source plants, preferably non-transformed wild-type plants. In an especially preferred embodiment the accumulation is increased 2-fold.

The starch content of plant parts, organs or tissues can be determined according to methods well-known to the person skilled in the art, e.g. according to the method described in the appended Examples or that of Morrell (Phytochemistry 25 (1986), 1579-1585).

As pointed out above, the transgenic plants of the invention may also show, preferably in addition to an elevated starch content, an increase in the amino acid content and in density of the respective starch storing organ or other part of the plant, preferably tuber, compared to the corresponding source plant. Such an increase in the amino acid content may lie in the range of at least 5%, advantageously of at least 10% and most preferred of at least 20% when looked at the overall amino acid content, i.e. the sum of the individual amino acids, preferably the usual proteinaceous amino acids, present in the plant tissue. Moreover, single amino acids, in particular essential amino acids, may show a corresponding or even stronger increase. In this regard, it is particularly preferred that, in the transgenic plants of the invention, the content of leucine, methionine and/or tryptophane, advantageously the content of all three of these amino acids, is increased when compared to the corresponding source plant. The increase of the density of a starch storing organ may lie at values of at least 0.5%, preferably of at least 1% of the density of the respective organ in the corresponding source plant.

Methods for determining the amino acid content and the density of a plant tissue are well known in the art and may, for instance, be carried out as exemplified in the appended Examples.

The reduction of endogenous ADK activity in the transgenic plants of the invention may result in a significant increase of yield in the starch storing parts, organs or tissues of the plant as defined by the overall fresh weight per plant. Preferably, this increase is to at least 110%, more preferably to at least 120%, still more preferably to at least 180% of the fresh weight per plant yielded from a corresponding wild-type plant. In particular, a transgenic plant of the present invention shows, if it is a tuber-bearing plant, preferably a potato plant, an increase in tuber yield, i.e. the overall weight of the produced tuber(s) per plant. The increase is preferably to at least 110%, more preferably at least 130%, even more preferably at least 150%, particularly preferred at least 160% and most preferably at least 180% of the tuber yield of corresponding wild-type plants, which are grown under the same environmental conditions. In addition, the transgenic plants of the invention may also show an increase in the number of starch storing organs such as tubers per plant, preferably to at least 110% of the number obtained from corresponding wild-type plants. Another important aspect of the increase in yield is the increase of the amount of starch by weight that is obtainable per plant. This yield parameter is preferably increased in the transgenic plants of the invention to at least 110% more preferably to at least 120%, still more preferably to at least 140% and most preferably to at least 180% of the amount of starch per plant yielded from a corresponding wild-type plant. In a further aspect, the fresh weight per harvestable starch-storing organ, e.g. tuber, may be significantly increased compared to corresponding wild-type plants, preferably to at least 120% and more preferably to at least 140% of the fresh weight of starch-storing organs from a corresponding wild-type plant.

In the context of the present invention, the term "transgenic" means that the plants contain cells in which the genome structurally deviates from that of the corresponding source plant in a way that the ADK activity is reduced as explained above. Such a structural difference preferentially refers to the gene encoding ADK, which may for instance be inactivated due to a deletion. The prior art provides means and methods for producing transgenic plants wherein the activity of a specific enzyme is reduced.

As a preferred embodiment, the transgenic plants of the present invention are characterized by the presence of a foreign nucleic acid molecule. Accordingly, the term "presence of a foreign nucleic acid molecule" as used herein refers to any nucleic acid molecule that is present in cells of a transgenic plant of the invention but absent from the cells of the corresponding source plant. Thereby encompassed are nucleic acid molecules, e.g. gene sequences, which differ from the corresponding nucleic acid molecule in the source plant cell by at least one mutation (substitution, insertion, deletion, etc. of at least one nucleotide), wherein such a mutation inhibits the expression of the affected gene or reduces the activity of the gene product. Furthermore encompassed by the term "foreign" are nucleic acid molecules which are homologous with respect to the source plant cell but are situated in a different chromosomal location or differ, e.g., by way of a reversed orientation for instance to the promoter.

In principle, the foreign nucleic acid molecule may be of any conceivable origin, e.g. eukaryotic or prokaryotic. It may be of any organism which comprises such molecules. Furthermore, it may be synthetic or derived from naturally occurring molecules by, e.g., modification of its sequence, i.e. it may be a variant or derivative of a naturally occurring molecule. Such variants and derivatives include but are not limited to molecules derived from naturally occurring molecules by addition, deletion, mutation of one or more nucleotides or by recombination. It is, e.g., possible to change the sequence of a naturally occurring molecule so as to match the preferred codon usage of plants, in particular of those plants in which the nucleic acid molecule shall be expressed.

The reduction of the endogenous ADK activity in the target plant by virtue of introducing a foreign nucleic acid molecule may be achieved by a suitable method known in the prior art, among these it is preferred to apply an antisense, co-suppression ribozyme or RNA interference effect or by in vivo mutagenesis, antibody expression or by the expression of a dominant-negative mutant. These methods are further explained in the following.

Accordingly, the use of nucleic acid molecules encoding an antisense RNA which is complementary to transcripts of a plant ADK is a preferred embodiment of the present invention. For expressing said antisense RNA, the nucleic acid molecule is operatively linked to a promoter allowing for expression is plants. The term "operative linked", as used throughout the present description, refers to a linkage between a promoter and the nucleic acid molecule to be expressed in such a way that expression is achieved under conditions compatible with the promoter. Thereby, complementarity does not signify that the encoded RNA has to be 100% complementary. A low degree of complementarity is sufficient, as long as it is high enough to inhibit the expression of an ADK upon expression of said RNA in plant cells. The transcribed RNA is preferably at least 90% and most preferably at least 95% complementary to the transcript of the nucleic acid molecule encoding ADK. In order to cause an antisense effect during the transcription in plant cells such RNA molecules have a length of at least 15 bp, preferably a length of more than 100 bp and most preferably a length or more than 500 bp, however, usually less than 5000 bp, preferably shorter than 2500 bp. Exemplary methods for achieving an antisense effect in plants are for instance described by Haake (Plant J. 14 (1998), 147-157), Tauberger (Plant J. 23 (2000), 43-53) and Tjaden (Plant J. 16 (1998), 531-540) and are herewith incorporated in the description of the present invention. Likewise, an antisense effect may also be achieved by applying a triple-helix approach, whereby a nucleic acid molecule complementary to a region of the ADK gene, designed according to the principles for instance laid down in Lee (Nucl. Acids Res. 6 (1979), 3073); Cooney (Science 241 (1998), 456) or Dervan (Science 251 (1991), 1360) may inhibit its transcription.

A similar effect as with antisense techniques can be achieved by producing transgenic plants expressing suitable constructs in order to mediate an RNA interference (RNAi) effect. Thereby, the formation of double-stranded RNA leads to an inhibition of gene expression in a sequence-specific fashion. More specifically, in RNAi constructs, a sense portion comprising the coding region of the gene to be inactivated (or a part thereof, with or without non-translated region) is followed by a corresponding antisense sequence portion. Between both portions, an intron not necessarily originating from the same gene may be inserted. After transcription, RNAi constructs form typical hairpin structures. In accordance with the teachings of the present invention, the RNAi technique may be carried out as described by Smith (Nature 407 (2000), 319-320) or Marx (Science 288 (2000), 1370-1372).

Also DNA molecules can be employed which, during expression in plant cells, lead to the synthesis of an RNA which reduces the expression of the nucleic acid molecules encoding ADK in the plant cells due to a co-suppression-effect. The principle of co-suppression as well as the production of corresponding DNA sequences is precisely described, for example, in WO 90/12084. Such DNA molecules preferably encode an RNA having a high degree of homology to transcripts of a gene encoding ADK. It is, however, not absolutely necessary that the coding RNA is translatable into a protein. The principle of the co-suppression effect is known to the person skilled in the art and is, for example, described in Jorgensen, Trends Biotechnol. 8 (1990), 340-344; Niebel, Curr. Top. Microbiol. Immunol. 197 (1995), 91-103; Flavell, Curr. Top. Microbiol. Immunol. 197 (1995), 43-36; Palaqui and Vaucheret, Plant. Mol. Biol. 29 (1995), 149-159; Vaucheret, Mol. Gen. Genet. 248 (1995), 311-317; de Borne, Mol. Gen. Genet. 243 (1994), 613-621 and in other sources.

Likewise, DNA molecules encoding an RNA molecule with ribozyme activity which specifically cleaves transcripts of a gene encoding ADK can be used. Ribozymes are catalytically active RNA molecules capable of cleaving RNA molecules and specific target sequences. By means of recombinant DNA techniques, it is possible to alter the specificity of ribozymes. There are various classes of ribozymes. For practical applications aiming at the specific cleavage of the transcript of a certain gene, use is preferably made of representatives of the group of ribozymes belonging to the group I intron ribozyme type or of those ribozymes exhibiting the so-called "hammerhead" motif as a characteristic feature. The specific recognition of the target RNA molecule may be modified by altering the sequences flanking this motif. By base pairing with sequences in the target molecule these sequences determine the position at which the catalytic reaction and therefore the cleavage of the target molecule takes place. Since the sequence requirements for an efficient cleavage are low, it is in principle possible to develop specific ribozymes for practically each desired RNA molecule. In order to produce DNA molecules encoding a ribozyme which specifically cleaves transcripts of a gene encoding an ADK, for example a DNA sequence encoding a catalytic domain of a ribozyme is bilaterally linked with DNA sequences which are complementary to sequences encoding the target protein ADK. Sequences encoding the catalytic domain may for example be the catalytic domain of the satellite DNA of the SCMo virus (Davies, Virology 177 (1990), 216-224 and Steinecke, EMBO J. 11 (1992), 1525-1530) or that of the satellite DNA of the TobR virus (Haseloff and Gerlach, Nature 334 (1988), 585-591). The expression of ribozymes in order to decrease the activity of certain proteins in cells is known to the person skilled in the art and is, for example, described in EP-B1 0 321 201. The expression of ribozymes in plant cells is for example described in Feyter (Mol. Gen. Genet. 250 (1996), 329-338).

Furthermore, the adenylate kinase activity in the plant cells of the invention can also be decreased by the so-called "in vivo mutagenesis", i.e. by methods where the sequence of an ADK encoding gene is modified at its natural chromosomal location such as for instance by techniques applying homologous recombination. This may be achieved by using a hybrid RNA-DNA oligonucleotide ("chimeroplast") which is introduced into cells by transformation (TIBTECH 15 (1997), 441-447; WO95/15972; Kren, Hepatology 25 (1997), 1462-1468; Cole-Strauss, Science 273 (1996), 1386-1389). Part of the DNA component of the RNA-DNA oligonucleotide is homologous to the target ADK gene sequence, however, displays in comparison to this sequence, a mutation or a heterologous region which is surrounded by the homologous regions. By means of base pairing of the homologous regions with the target sequence followed by a homologous recombination, the mutation or the heterologous region contained in the DNA component of the RNA-DNA oligonucleotide can be transferred to the corresponding gene of the plant cell. By means of in vivo mutagenesis, any part of the ADK-encoding gene can be inactivated as long as it results in a decrease of the endogenous ADK activity. Thus, this can concern for instance, the promoter, e.g. the RNA polymerase binding site, as well as the coding region, in particular those parts encoding the catalytically active center or a signal sequence directing the protein to the appropriate cellular compartment. Further methods for obtaining transgenic plants wherein the respective endogenous ADK-encoding gene is inactivated include screening methods of libraries of transgenic plant lines containing randomized knock-out mutations as for instance introduced by T-DNA or transposon-tagging. Preferentially, such screenings are based on the genotype, i.e. by identifying lines in which the structure of an ADK-encoding gene deviates from the wild-type locus. Suitable methods are described in literature such as in Kumar (Meth. Enzymology 328 (2000), 550-574).

Furthermore, nucleic acid molecules encoding antibodies specifically recognizing ADK in a plant, i.e. specific fragments or epitopes of such a protein, can be used for inhibiting the activity of this protein. These antibodies can be monoclonal antibodies, polyclonal antibodies or synthetic antibodies as well as fragments of antibodies, such as Fab, Fv or scFv fragments etc. Monoclonal antibodies can be prepared, for example, by the techniques as originally described in Kohler and Milstein (Nature 256 (1975), 495) and Galfro (Meth. Enzymol. 73 (1981) 3), which comprise the fusion of mouse myeloma cells to spleen cells derived from immunized mammals. Furthermore, antibodies or fragments thereof to the aforementioned peptides can be obtained by using methods which are described, e.g., in Harlow and Lane "Antibodies, A Laboratory Manual", CSH Press, Cold Spring Harbor, 1988. Expression of antibodies or antibody-like molecules in plants can be achieved by methods well known in the art, for example, full-size antibodies (Daring, Plant. Mol. Biol. 15 (1990), 281-293; Hiatt, Nature 342 (1989), 469-470; Voss, Mol. Breeding 1 (1995), 39-50), Fab-fragments (De Neve, Transgenic Res. 2 (1993), 227-237), scFvs (Owen, Bio/Technology 10 (1992), 790-794; Zimmermann, Mol. Breeding 4 (1998), 369-379; Tavladoraki, Nature 366 (1993), 469-472) and dAbs (Benvenuto, Plant Mol. Biol. 17 (1991), 865-874) have been successfully expressed in tobacco, potato (Schouten, FEBS Lett. 415 (1997), 23Arabidopsis, reaching expression levels as high as 6.8% of the total protein (Fiedler, Immunotechnology 3 (1997), 205-216).

In addition, nucleic acid molecules encoding a mutant form of ADK can be used to interfere with the activity of the wild-type protein. Such a mutant form preferably has lost its biological activity, e.g. kinase activity, and may be derived from the corresponding wild-type protein by way of amino acid deletion(s), substitution(s), and/or additions in the amino acid sequence of the protein. Mutant forms of such proteins may show, in addition to the loss of kinase activity, an increased substrate affinity and/or an elevated stability in the cell, for instance, due to the incorporation of amino acids that stabilize proteins in the cellular environment. These mutant forms may be naturally occurring or, as preferred, genetically engineered mutants.

Furthermore, it is immediately evident to the person skilled in the art that the above-described antisense, ribozyme, RNA interference, co-suppression, in-vivo mutagenesis, antibody expression and dominant mutant effects can also be used for the reduction of the expression of genes that encode a regulatory protein such as transcription factors, that control the expression of ADK or, e.g., proteins that are necessary for ADK to become active.

It is also evident from the disclosure of the present invention that any combination of the above-identified strategies can be used for the generation of transgenic plants, which due to the one or more of the above-described foreign nucleic acid molecules in their cells display a reduced ADK activity compared to the corresponding source plant. Such combinations can be made, e.g., by (co-)transformation of corresponding nucleic acid molecules into the plant cell, plant tissue or plant or by crossing transgenic plants that have been generated by different embodiments of the above-described method of the present invention. Likewise, the plants obtainable by the method of the present invention can be crossed with other transgenic plants so as to achieve a combination of increased starch accumulation and another genetically engineered trait, such as for example stress tolerance or a modified starch biosynthesis.

For some of the above-described embodiments, the foreign nucleic acid molecule is expressed in the transgenic plant of the invention, whereby the term "expressed" means that the nucleic acid molecule is at least transcribed, and for some embodiments also translated into a protein, in at least some of the cells of the plant. It is in principle possible that the foreign nucleic acid molecule is expressed in all or substantially all cells of the plant. However, it is also possible that it is only expressed in certain parts, organs, cell types, tissues etc. Moreover, it is possible that the expression of the foreign nucleic acid molecule only takes place upon induction or only at a certain developmental stage. In a preferred embodiment, the nucleic acid is expressed in a starch-storing organ or tissue, e.g. in the potato tuber.

In order to be expressed, the foreign nucleic molecule contained in the transgenic plant according to the invention is preferably linked to a promoter allowing expression in plant cells.

The promoter may be homologous or heterologous to the plant. Suitable promoters are for instance the promoter of the 35S RNA of the Cauliflower Mosaic Virus (see for instance US-A-5,352,605) and the ubiquitin-promoter (see for instance US-A-5,614,399) which lend themselves to constitutive expression, the patatin gene promoter B33 (Rocha-Sosa et al., EMBO J. 8 (1989), 23-29) which lends itself to a tuber-specific expression in potatoes or a promoter ensuring expression in photosynthetically active tissues only, for instance the ST-LS1 promoter (Stockhaus et al., Proc. Natl. Acad. Sci. USA 84 (1987), 7943-7947; Stockhaus et al., EMBO, J. 8 (1989) 2445-2451), the Ca/b-promoter (see for instance US-A-5,656,496, US-A-5,639,952, Bansal et al., Proc. Natl. Acad. Sci. USA 89 (1992), 3654-3658) and the Rubisco SSU promoter (see for instance US-A-5,034,322; US-A-4,962,028) or the glutelin promoter from wheat which lends itself to endosperm-specific expression (HMW promoter) (Anderson, Theoretical and Applied Genetics 96, (1998), 568-576, Thomas, Plant Cell 2 (12), (1990), 1171-1180), the glutelin promoter from rice (Takaiwa, Plant Mol. Biol. 30(6) (1996), 1207-1221, Yoshihara, FEBS Lett. 383 (1996), 213-218, Yoshihara, Plant and Cell Physiology 37 (1996), 107-111), the shrunken promoter from maize (Maas, EMBO J. 8 (11) (1990), 3447-3452, Werr, Mol. Gen. Genet. 202(3) (1986), 471-475, Werr, Mol. Gen. Genet. 212(2), (1988), 342-350), the USP promoter, the phaseolin promoter (Sengupta-Gopalan, Proc. Natl. Acad. Sci. USA 82 (1985), 3320-3324, Bustos, Plant Cell 1 (9) (1989), 839-853) or promoters of zein genes from maize (Pedersen et al., Cell 29 (1982), 1015-1026; Quatroccio et al., Plant Mol. Biol. 15 (1990), 81-93). However, promoters which are only activated at a point in time determined by external influences can also be used (see for instance WO 93/07279). In this connection, promoters of heat shock proteins which permit simple induction may be of particular interest. Moreover, seed-specific promoters such as the USP promoter from Vicia faba which ensures a seed-specific expression in Vicia faba and other plants may be used (Fiedler et al., Plant Mol. Biol. 22 (1993), 669-679; Baumlein et al., Mol. Gen. Genet. 225 (1991), 459-467). Moreover, fruit-specific promoters, such as described in WO 91/01373 may be used too. Preferred are promoters which ensure constitutive expression.

Moreover, the foreign nucleic acid molecule may be linked to a termination sequence, which serves to terminate transcription correctly and to add a poly-A-tail to the transcript, which is believed to have a function in the stabilization of the transcripts. Such elements are described in the literature (see for instance Gielen et al., EMBO J. 8 (1989), 23-29) and can be replaced at will.

If the foreign nucleic acid molecule is expressed in the transgenic plants according to the invention to produce a polypeptide, there exists in principle the possibility that the synthesized protein can be localized in any compartment of the plant cell (e.g. in the cytosol, plastids, vacuole, mitochondria) or the plant (e.g. in the apoplast). In order to achieve the localization in a particular compartment, the coding region must, where necessary, be linked to DNA sequences ensuring localization in the corresponding compartment. The signal sequences used must each be arranged in the same reading frame as the DNA sequence encoding the enzyme. The localization in the plastids is preferred.

In order to ensure the location in the plastids it is conceivable to use one of the following transit peptides: of the plastidic Ferredoxin: NADP+ oxidoreductase (FNR) of spinach which is enclosed in Jansen et al. (Current Genetics 13 (1988), 517-522). In particular, the sequence ranging from the nucleotides -171 to 165 of the cDNA Sequence disclosed therein can be used which comprises the 5 non-translated region as well as the sequence encoding the transit peptide. Another example is the transit peptide of the waxy protein of maize including the first 34 amino acid residues of the mature waxy protein (Klosgen et al., Mol. Gen. Genet. 217 (1989), 155-161). It is also possible to use this transit peptide without the first 34 amino acids of the mature protein. Furthermore, the signal peptides of the ribulose bisphosphate carboxylase small subunit (Wolter et al., Proc. Natl. Acad. Sci. USA 85 (1988), 846-850; Nawrath et al., Proc. Natl. Acad. Sci. USA 91 (1994), 12760-12764), of the NADP malat dehydrogenase (Gallardo et al., Planta 197 (1995), 324-332), of the glutathion reductase (Creissen et al., Plant J. 8 (1995), 167-175) or of the R1 protein (Lorberth et al. Nature Biotechnology 16, (1998), 473-477) can be used. Another suitable signal peptide for plastid-directed transfer is that of StpADK comprising the amino acid sequence from position 1 to 78 of SEQ ID NO: 2.

In order to ensure the location in the vacuole, it is conceivable to use one of the following transit peptides: the N-terminal sequence (146 amino acids) of the patatin protein (Sonnewald et al., Plant J. 1 (1991), 95-106) or the signal sequences described by Matsuoka and Neuhaus (Journal of Experimental Botany 50 (1999), 165-174); Chrispeels and Raikhel (Cell 68 (1992), 613-616); Matsuoka and Nakamura (Proc. Natl. Acad. Sci. USA 88 (1991), 834-838); Bednarek and Raikhel (Plant Cell 3 (1991), 1195-1206); and Nakamura and Matsuoka (Plant Phys. 101 (1993), 1-5).

In order to ensure the location in the mitochondria, it is for example conceivable to use the transit peptide described by Braun (EMBO J. 11, (1992), 3219-3227).

In order to ensure the location in the apoplast, it is conceivable to use one of the following transit peptides: signal sequence of the proteinase inhibitor II-gene (Keil et al., Nucleic Acid Res. 14 (1986), 5641-5650; von Schaewen et al., EMBO J. 9 (1990), 30-33), of the levansucrase gene from Erwinia amylovora (Geier and Geider, Phys. Mol. Plant Pathol. 42 (1993), 387-404), of a fragment of the patatin gene B33 from Solanum tuberosum, which encodes the first 33 amino acids (Rosahl et al., Mol Gen. Genet. 203 (1986), 214-220) or of the one described by Oshima et al. (Nucleic Acid Res. 18 (1990), 181).

The transgenic plants according to the invention may, in principle, be plants of any plant species, that is to say they may be monocotyledonous and dicotyledonous plants. Preferably, the plants are useful plants cultivated by man for nutrition or for technical, in particular industrial, purposes. They are preferably starch-storing plants, for instance cereal species (rye, barley, oat, wheat, millet, sago etc.), rice, pea, marrow pea, cassava and potato; tomato, rape, soybean, hemp, flax, sunflower, cow pea or arrowroot, fiber-forming plants (e.g. flax, hemp, cotton), oil-storing plants (e.g. rape, sunflower, soybean) and protein-storing plants (e.g. legumes, cereals, soybeans). The invention also relates to fruit trees and palms. Moreover, the invention relates to forage plants (e.g. forage and pasture grasses, such as alfalfa, clover, ryegrass) and vegetable plants (e.g. tomato, lettuce, chicory) and ornamental plants (e.g. tulips, hyacinths). Sugar-storing and/or starch-storing plants are preferred. Sugar cane and sugar beet, maize, rice, wheat and tomato plants are particularly preferred, and potato plants most preferred.

The transgenic plants according to the invention can be prepared by introducing a foreign nucleic acid molecule into plant cells and regenerating the transformed cells to plants by methods well known to the person skilled in the art.

A plurality of techniques is available by which DNA can be inserted into a plant host cell. These techniques include the transformation of plant cells by T-DNA using Agrobacterium tumefaciens or Agrobacterium rhizogenes as a transforming agent, the fusion of protoplasts, injection, electroporation of DNA, insertion of DNA by the biolistic approach and other possibilities.

The use of the Agrobacteria-mediated transformation of plant cells has been extensively investigated and sufficiently described in EP 120 516; Hoekema, In: The Binary Plant Vector System, Offsetdrukkerij Kanters B.V., Alblasserdam (1985), Chapter V; Fraley et al, Crit. Rev. Plant Sci. 4 (1993), 1-46 and An et al., EMBO J. 4 (1985), 277-287. Regarding the transformation of potatoes see for instance Rocha-Sosa et al. (EMBO J. 8 (1989), 29-33).

The transformation of monocotyledonous plants by means of Agrobacterium-based vectors has also been described (Chan et al., Plant Mol. Biol. 22 (1993), 491-506; Hiei et al., Plant J. 6 (1994) 271-282; Deng et al, Science in China 33 (1990), 28-34; Wilmink et al, Plant Cell Reports 11 (1992), 76-80; May et al., Bio/Technology 13 (1995), 486-492; Conner and Dormisse, Int. J. Plant Sci. 153 (1992), 550-555; Ritchie et al. Transgenic Res. 2 (1993), 252-265). An alternative system for transforming monocotyledonous plants is the transformation by the biolistic approach (Wan and Lemaux, Plant Physiol. 104 (1994), 37-48; Vasil et al., Bio/Technology 11 (1993), 1553-1558; Ritala et al., Plant Mol. Biol. 24 (1994) 317-325; Spencer et al., Theor. Appl. Genet. 79 (1990), 625-631), protoplast transformation, electroporation of partially permeabilized cells, insertion of DNA via glass fibers. The transformation of maize in particular has been repeatedly described in the literature (see for instance WO 95/06128, EP 0 513 849, EP 0 465 875, EP 29 24 35; Fromm et al, Biotechnology 8, (1990), 833-844; Gordon-Kamm et al., Plant Cell 2, (1990), 603-618; Koziel et al., Biotechnology 11 (1993), 194-200; Moroc et al., Theor. Appl. Genet. 80, (1990), 721-726). The successful transformation of other types of cereals has also been described for instance of barley (Wan and Lemaux, supra; Ritala et al., supra, Krens et al., Nature 296 (1982), 72-74) and wheat (Nehra et al., Plant J. 5 (1994), 285-297).

The present invention also relates to transgenic plant cells which preferably are contained in a transgenic plant according to the invention, said cells being characterized by having a reduced endogenous ADK activity. As regards, the features and modes for producing such transgenic cells, the same explanations and embodiments apply as those specified for the transgenic plants herein above.

The invention also relates to propagation material of the plants of the invention comprising plant cells according to the invention. The term "propagation material" comprises those components or parts of the plant which are suitable to produce offspring vegetatively or generatively. Suitable means for vegetative propagation are for instance cuttings, callus cultures, rhizomes or tubers. Other propagation material includes for instance fruits, seeds, seedlings, protoplasts, cell cultures etc. The preferred propagation materials are tubers and seeds. The invention also relates to harvestable parts of the plants of the invention such as, for instance, fruits, seeds, tubers or rootstocks.

The present invention also relates to a recombinant nucleic acid molecule comprising: (a) a promoter ensuring transcription in plant cells; and operatively linked thereto (b) a nucleic acid sequence which, when transcribed in plant cells, leads to a reduction of the endogenous ADK activity in said plant cells; and optionally (c) a transcription termination signal.

With regard to preferred embodiments concerning the promoter, the transcription termination signal, the nucleic acid sequence mentioned under (b) and the sub-cellular targeting of the protein encoded by said nucleic acid molecule, the same applies what was already mentioned above in connection with the transgenic plants according to the invention.

The present invention also relates to vectors containing a recombinant nucleic acid molecule of the invention. Preferred are vectors which allow for the transformation of plant cells and most preferred are vectors which allow for the stable integration of the nucleic acid molecule into the plant genome, such as e.g. binary vectors. Such vectors are extensively described in the literature and are also commercially available.

Moreover, the present invention relates to the use of a recombinant nucleic acid molecule according to the invention for the preparation of transgenic plants containing and expressing a foreign nucleic acid molecule which, when transcribed in plant cells, leads to a reduction of the endogenous ADK mRNA level in said plant cells.

The present invention furthermore relates to a process for producing transgenic plants displaying an increase in starch accumulation and/or in yield of starch storing parts, organs or tissues comprising the steps of: (a) introducing into a plant cell a nucleic acid molecule the presence of which in the genome of said plant leads to a reduced endogenous ADK activity in plant cells; (b) regenerating from transformed cells produced in step (a) plants; and optionally (c) producing progeny from the transgenic plants produced in step (b).

The term "increase in yield of starch-storing parts, organs or tissues" refers to any of the aforementioned yield-related improvements that can be attained according to the provisions of the present invention, in particular to the increase in yield of starch-storing parts, organs or tissues, especially tubers or kernels, as defined by fresh weight per plant, in the number of starch-storing organs, in the amount of starch by weight per plant or in the fresh weight of each harvestable starch-storing organ or in any combination of any one of these aspects.

With respect to step (a), the nucleic acid molecule to be introduced may be a recombinant nucleic acid molecule or a vector according to the invention as described herein above.

Step (b) can be carried out according to methods well-known to the person skilled in the art.

The production of progeny according to step (c) of the process includes vegetative as well as sexual propagation.

The present invention also relates to transgenic plants obtained or obtainable by the process according to the invention.

In addition, the present invention pertains to the use of a recombinant nucleic acid molecule or a vector as described above for the production of transgenic plants or transgenic plant cells displaying an increase in starch accumulation and/or in yield of starch storing parts, organs or tissues.

These and other embodiments are disclosed and obvious to a skilled person and embraced by the description and the examples of the present invention. Additional literature regarding one of the above-mentioned methods, means and applications, which can be used within the meaning of the present invention, can be obtained from the state of the art, for instance from public libraries for instance by the use of electronic means. This purpose can be served inter alia by public databases, such as the "medline", which are accessible via internet, for instance under the address

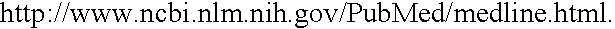

Other databases and addresses are known to a skilled person and can be obtained from the internet, for instance under the address

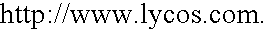

An overview of sources and information regarding patents and patent applications in biotechnology is contained in Berks, TIBTECH 12 (1994), 352-364.

Furthermore, the term "and/or" whenever occurring herein includes the meaning of "and", "or" and "all or any other combination of the elements connected by said term".

All of the above cited disclosures of patents, publications and database entries are specifically incorporated herein by reference in their entirety to the same extent as if each such individual patent, publication or entry were specifically and individually indicated to be incorporated by reference.

The following Examples serve to further illustrate the invention.

In the Examples the following materials and methods were used.

1.Molecular biological techniques Unless stated otherwise in the examples, all recombinant DNA techniques are performed according to protocols as described in Sambrook et al. (1989), Molecular Cloning : A Laboratory Manual. Cold Spring Harbor Laboratory Press, NY or in Volumes 1 and 2 of Ausubel et al. (1994), Current Protocols in Molecular Biology, Current Protocols. Standard materials and methods for plant molecular work are described in Plant Molecular Biology Labfase (1993) by R.D.D. Croy, jointly published by BIOS Scientific Publications Ltd (UK) and Blackwell Scientific Publications (UK).

2.Plant Material Potato plants (Solanum tuberosum L. cv. Desiree obtained from Saatzucht Lange AG, Bad Schwartau, Germany) were maintained in tissue culture with a 16-h light, 8-h dark regime on MS medium (Murashige and Skoog, Physiologia Plantarum 15 (1962), 473-497) which contained 2% sucrose. In the greenhouse, plants were grown under the same light regime with a minimum of 250 mol photons m-2 s-1at 22 C. In connection with the present invention, the term "developing tubers" is used for tubers (over 10 g FW) harvested from healthy 10-week old plants; "mature tubers" is used to refer to tubers harvested from senescent plants.

3.Preparation of transgenic antisense lines In order to get hold of a cDNA encoding the potato plastidial adenylate kinase, a potato tuber cDNA library which has been prepared as described in Kossmann (Mol. Gen. Genet. 230 (1992), 39-44) was screened with a probe derived from the corresponding maize gene (Shen, Plant Mol. Biol. 26 (1994), 1085-1101). The probe was prepared by PCR amplification from genomic maize DNA using a 40 cycle program with an annealing temperature of 40 C and the primers M-AdK5' (SEQ ID NO:3) and M-AdK3' (SEQ ID NO:4) resulting in a 400 bp fragment as expected from the corresponding maize cDNA sequence (Genbank/EMBL database entry T25266). Screening of the cDNA library was performed using Denhardt's buffer at 65 C, for 3h, washed, positive phages were streaked out and clones were in vivo excised. These were then digested by EcoRI to identify true positives. A positive cDNA clone obtained by the screening contains a fragment of about 900 bp (SEQ ID NO: 1) cloned into the EcoRI/XhoI sites of pBluescriptSK which was identified by homology with the maize gene to encode a plastidial adenylate kinase.

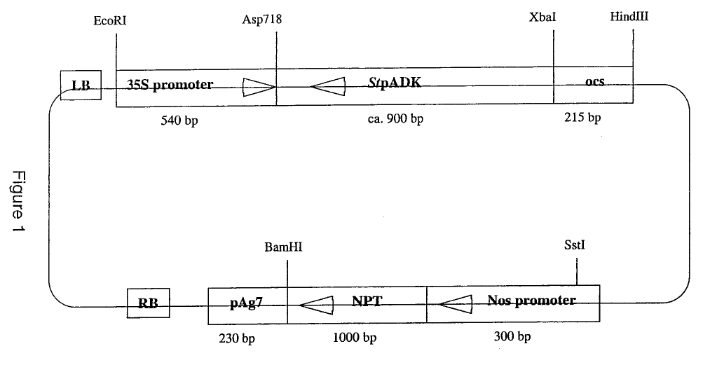

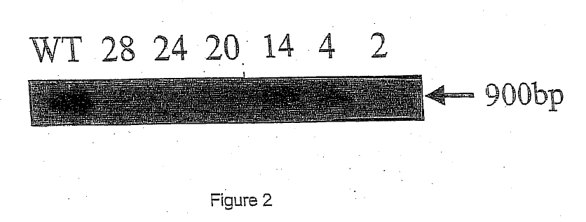

This cDNA fragment was excised at the Asp 718/XbaI sites and ligated into the corresponding restriction sites of the vector pBinAR-Kan (Liu et al., Molecular and General Genetics 223 (1990), 401-406) between the CaMV 35S promoter and the ocs terminator as shown in Figure 1. This construct was introduced into potato plants by applying an Agrobacterium-mediated transformation protocol (Rocha-Sosa et al., EMBO J. 8 (1989), 23-29). Transgenic plants were selected on kanamycin-containing medium (Dietz et al., 1995, In: Gene transfer to plants XXII, Potrykus, I. and Spangenberg, G., eds., Berlin, Springer-Verlag, pp.24-29). Initial screening of around 80 lines was performed by determining the specific density and yield of tubers harvested plants grown in 3.5 litre pots under greenhouse conditions. A second screen was then performed at the transcript and enzyme activity levels with tubers and leaves from six plants per line for the nine lines initially selected in the greenhouse. Transcript determination (Figure 2) was carried out using the riboprobe method as described in Schenborn (Nucl. Acids Res. 13 (1985), 6223), Krieg (Meth. Enzymol. 155 (1987), 397) and the Promega technical manual #016.

4.Biochemical analysis Starch, sugars, amino acids and glycolytic metabolites were determined exactly as described in Trethewey et al. (Plant Cell and Environment 22 (1999), 71-79) whilst nucleotides were assayed using an HPLC system as detailed in Geigenberger et al. (Planta 205 (1999), 428-437). In the repeated analysis (Example 2) starch, sugars, glycolytic metabolites and nucleotides were determined exactly as described in Geigenberger et al. (Planta 205 (1998), 428-437), amino acids as in Geigenberger et al. (Plant Cell and Environment 19 (1996), 43-55). Adenylate kinase activity was measured by the protocol of Kleczkowski and Randall (Plant Physiol. 81 (1986), 1110-1114) in leaf and tuber samples and in leaf chloroplasts preparations that had been isolated as detailed by Tauberger et al. (Plant J. 23 (2000), 43-53). Contamination of these preparations by cytosolic marker enzymes did not exceed 10% in either wild type or transgenic tissue. Other enzymes of starch synthesis were measured as detailed in Fernie et al. (Planta 213 (2001), 418-426).

Example 1 Morphological and biochemical analysis of transgenic ADK antisense potato plants. The aim of the present work was to establish the importance of the plastidial adenylate kinase to biosynthetic pathways within the potato tuber amyloplast. For this purpose the cDNA encoding the plastidial isoform of potato adenylate kinase (StpADK) has been cloned (SEQ ID NO: 1 having the deduced amino acid sequence shown in SEQ ID NO: 2). It possesses a functional plastid targeting sequence (position 1 to 78 of SEQ ID NO: 2). Using the antisense approach, transgenic plants were generated as described above that exhibited a decreased expression of the StpADK gene. Accordingly, these lines showed a significantly reduced total adenylate kinase activity (down to 75% of wild-type activity). Furthermore, it could be demonstrated that the loss in activity was localized to the plastid. Transformant lines with decreased activities of plastidial ADK exhibited in general no major changes in morphology. However, the tuber morphology of the transgenic plants deviates from that of the wild-type plants. In particular, the density of the tubers is significantly increased over those of the wild-type plants (see Table 1).

When analyzing the metabolites present in the transgenic tubers, a remarkable increase of the starch content is apparent (Table 2). All of the transgenic antisense lines under investigation significantly show an elevated accumulation of starch measured in mol glucose relative to fresh weight (FW), which ranges up to 162% above that of wild-type tubers. Furthermore, the content of the intermediate metabolites of the carbohydrate synthesis pathways UDP-glucose, glucose-6-phospate, 3-phosphoglycerate, triose-phosphate and ADP-glucose as well as that of adenylates has been dertermined (Table 3).

Most remarkably, the total adenylate (ANT) content is considerably increased, a result which, in view of the reaction catalyzed by ADK, could not be expected. Similarly, the observed decrease of the ATP/ADP ratio in the transgenic tubers was not foreseeable. As a further consequence of the reduced ADK activity, the amino acid content is also distinctly elevated in the transgenic tubers (Table 4). All of the transgenic plants under investigation showed a significant increase of the total amino acid content. Some amino acids, among those essential amino acids such as leucine, methionine and tryptophane, are especially elevated, while others remain in an equivalent concentration range as compared to wild-type tubers. As a conclusion, it was found that starch and amino acid content strongly increases in plants where the activity of the plastidial ADK is repressed. Both, starch and amino acid biosynthesis are heavily reliant on ATP supply to the plastid and these results therefore suggest that adenylate kinase also acts in the ATP consuming direction in vivo. One may therefore conclude that plastidial adenylate kinase must represent an energy constraint on plastidial biosynthesis.

TABLE 1
Yield, density, mean tuber size and number of the antisense StpAdK transgenic lines.

| Parameter | WT | Adk-2 | AdK-4 | Adk-14 | AdK-20 | AdK-24 | AdK-28 |
|---|---|---|---|---|---|---|---|
| Tuber Number | 10.3 ± 0.8 | 9.0 ± 0.4 | 8.8 ± 1.9 | 18.0^{¶} | 12.0 ± 1.5 | 14.0 ± 3.8 | 7.5 ± 1.1 |
| Tuber yield | 365 ± 43 | 357 ± 22 | 324 ± 37 | 544^{¶} | 310 ± 25 | 377 ± 34 | 275 ± 30 |
| Mean tuber FW(g) | 35.4 | 39.7 | 36.8 | 30.2 | 25.8 | 26.9 | 36.7 |
| Density | 1.086 ± 0.006 | 1.093 ± | 1.098 ± | 1.091 ± | 1.096 ± | 1.091 ± | 1.091 ± |
| | | 0.004 | 0.002 | 0.001 | 0.001 | 0.005 | 0.003 |

Potato plants were grown in the greenhouse in 3.5 litre pots.
Transgenic tuber yield (total tuber fresh weight), tuber number and density determinations for mature tubers from fully senescent plants were performed with 10-15 plants per line harvested in the spring.
Values are mean ± SE.
^{¶indicates that only one plant was analysed for line AdK-14 in this trial}

TABLE 2
Carbohydrate content of the antisense StpADK transgenic tubers.

| | WT | AdK-2 | AdK-4 | Adk-14 | AdK-20 | AdK-24 | AdK-28 |
|---|---|---|---|---|---|---|---|
| Starch | 471 ± 48 | 678 ± 81 | 632 ± 63 | 693 ± 37 | 590 ± 38 | 752 ± 47 | 588 ± 48 |

Potato plants were grown in the greenhouse in 2.5 litre pots.
Developing tubers were harvested in the autumn after 10 weeks of growth and the sucrose, glucose and starch content were determined.
Data are presented in μmol glucose gFW^{-1 and represent the mean ± SE of determinations on six individual plants per line.}

TABLE 3
Metabolite content of the antisense StpADK transgenic tubers.

| | WT | AdK-2 | AdK-4 | Adk-14 | AdK-20 | AdK-24 | AdK-28 |
|---|---|---|---|---|---|---|---|
| nmol gFW^{-1 min}⁻¹ | | | | | | | |
| UDPglc | 88 ± 2 | 85 ± 11 | 71 ± 10 | 93 ± 6 | 86 ± 5 | 89 ± 9 | 74 ± 5 |
| Glc-6-P | 139 ± 8 | 113 ± 6 | 101 ± 6 | 131 ± 7 | 131 ± 6 | 148 ± 12 | 75 ± 7 |
| 3-PGA | 83 ± 13 | 69 ± 11 | 71 ± 11 | 76 ± 14 | 68 ± 10 | 105 ± 17 | 80 ± 13 |
| Triose-P | 5.4 ± 1.2 | 4.0 ± 0.5 | 2.6 ± 0.4 | 4.6 ± 1.5 | 2.2 ± 0.3 | 3.2 ± 0.4 | 4.3 ± 0.8 |
| ATP | 24 ± 3 | 32 ± 4 | 29 ± 4 | 33 ± 2 | 21 ± 1 | 28 ± 4 | 26 ± 3 |
| ADP | 13 ± 1 | 24 ± 5 | 25 ± 2 | 25 ± 2 | 19 ± 1 | 20 ± 1 | 26 ± 2 |
| AMP | 9.1 ± 0.9 | 13.8 ± 1.6 | 13.2 ± 1.2 | 17.3 ± 2.3 | 14.2 ± 1.1 | 13.9 ± 1.7 | 12.4 ± 0.9 |
| Total ANTs | 46.7 ± 2.4 | 69.6 ± 6.5 | 70.0 ± 4.1 | 75.2 ± 3.4 | 54.6 ± 2.2 | 62.1 ± 2.8 | 63.8 ± 3.9 |
| ADPglc | 0.10 ± 0.02 | 1.09 ± 0.29 | 1.02 ± 0.24 | 0.32 ± 0.11 | 0.41 ± 0.10 | 0.49 ± 0.10 | 0.61 ± 0.12 |
| Ratio | | | | | | | |
| ATP/ADP | 2.02 ± 0.37 | 1.50 ± 0.24 | 1.06 ± 0.12 | 1.40 ± 0.12 | 1.18 ± 0.02 | 1.46 ± 0.26 | 1.06 ± 0.17 |
| Energy charge | 0.66 ± 0.03 | 0.62 ± 0.03 | 0.61 ± 0.03 | 0.61 ± 0.03 | 0.57 ± 0.01 | 0.61 ± 0.04 | 0.61 ± 0.02 |
| ADK constant | 0.80 ± 0.21 | 1.46 ± 0.53 | 2.10 ± 0.19 | 1.16 ± 0.19 | 1.15 ± 0.14 | 1.26 ± 0.31 | 2.15 ± 0.26 |

Potato plants were grown in the greenhouse in 2.5 litre pots.
Developing tubers were harvested in the autumn after 10 weeks of growth and the metabolite contents were determined.
Data are presented in nmol gFW^{-1 and represent the mean ± SE of determinations on six individual plants per line.}

TABLE 4
Amino acid content of the antisense StpADK transgenic tubers.

| (umol g FW⁻¹⁾ | WT | AdK-2 | AdK-4 | Adk-14 | AdK-20 | AdK-24 | AdK-28 |
|---|---|---|---|---|---|---|---|
| Alanine | 0.65 ± 0.08 | 1.35 ± 0.38 | 1.03 ± 0.04 | 1.17 ± 0.48 | 1.07 ± 0.2 | 0.80 ± 0.49 | 1.12 ± 0.17 |
| Arginine | 2.55 ± 0.43 | 4.01 ± 0.34 | 3.47 ± 0.2 | 3.18 ± 0.82 | 3.54 ± 0.80 | 3.85 ± 0.60 | 2.81 ± 0.11 |
| Asparagine | 28.3 ± 1.05 | 32.8 ± 3.55 | 32.6 ± 3.03 | 33.1 ± 8.7 | 37.7 ± 10.1 | 33.7 ± 5.1 | 36.7 ± 5.21 |
| Aspartate | 2.10 ± 0.07 | 1.87 ± 0.25 | 2.17 ± 0.23 | 1.99 ± 0.41 | 1.89 ± 0.46 | 2.07 ± 0.25 | 1.96 ± 0.11 |
| Glutamate | 3.89 ± 0.40 | 4.57 ± 0.54 | 5.14 ± 0.48 | 4.90 ± 0.89 | 4.64 ± 0.81 | 4.64 ± 0.81 | 5.03 ± 0.33 |
| Glutamine | 9.21 ± 1.04 | 9.24 ± 0.95 | 10.5 ± 1.63 | 8.27 ± 2.61 | 8.30 ± 2.19 | 8.64 ± 1.71 | 8.65 ± 1.03 |
| Glycine | 0.25 ± 0.02 | 0.32 ± 0.04 | 0.29 ± 0.02 | 0.30 ± 0.05 | 0.28 ± 0.04 | 0.28 ± 0.04 | 0.26 ± 0.02 |
| Histidine | 0.69 ± 0.06 | 1.00 ± 0.11 | 0.91 ± 0.09 | 0.76 ± 0.21 | 1.08 ± 0.23 | 1.02 ± 0.02 | 0.73 ± 0.02 |
| Isoleucine | 1.06 ± 0.04 | 1.74 ± 0.02 | 1.31 ± 0.12 | 1.09 ± 0.29 | 1.76 ± 0.37 | 1.67 ± 0.28 | 1.27 ± 0.09 |
| Leucine | 0.22 ± 0.04 | 0.90 ± 0.17 | 0.47 ± 0.08 | 0.38 ± 0.07 | 0.70 ± 0.22 | 0.87 ± 0.20 | 0.45 ± 0.08 |
| Methionine | 0.85 ± 0.05 | 1.44 ± 0.12 | 1.17 ± 0.13 | 1.22 ± 0.34 | 1.37 ± 0.26 | 1.29 ± 0.19 | 1.51 ± 0.18 |
| Phenylalanine | 0.79 ± 0.03 | 1.45 ± 0.21 | 1.07 ± 0.12 | 0.98 ± 0.27 | 1.51 ± 0.35 | 1.42 ± 0.28 | 1.21 ± 0.09 |
| Lysine | 1.22 ± 0.15 | 2.28 ± 0.20 | 1.48 ± 0.17 | 1.25 ± 0.29 | 2.03 ± 0.41 | 2.16 ± 0.35 | 1.65 ± 0.14 |
| Serine | 0.72 ± 0.06 | 1.10 ± 0.17 | 0.94 ± 0.07 | 0.73 ± 0.17 | 0.84 ± 0.17 | 0.93 ± 0.11 | 1.04 ± 0.07 |
| Threonine | 0.47 ± 0.04 | 0.44 ± 0.04 | 0.48 ± 0.03 | 0.41 ± 0.10 | 0.50 ± 0.10 | 0.46 ± 0.07 | 0.45 ± 0.02 |
| Tryptophan | 0.09 ± 0.04 | 0.38 ± 0.06 | 0.31 ± 0.05 | 0.27 ± 0.05 | 0.28 ± 0.09 | 0.39 ± 0.06 | 0.26 ± 0.15 |
| Tyrosine | 0.66 ± 0.08 | 1.18 ± 0.13 | 0.79 ± 0.05 | 0.57 ± 0.14 | 1.06 ± 0.25 | 1.10 ± 0.18 | 0.68 ± 0.04 |
| Valine | 2.77 ± 0.22 | 3.76 ± 0.43 | 3.12 ± 0.26 | 2.70 ± 0.70 | 3.68 ± 0.73 | 3.79 ± 0.60 | 2.90 ± 0.11 |
| Gaba | 5.88 ± 0.41 | 5.08 ± 0.84 | 5.50 ± 0.25 | 4.91 ± 1.35 | 5.78 ± 0.88 | 4.73 ± 0.57 | 4.65 ± 0.3 |
| Total amino acids | 62.4 ± 3.8 | 75.0 ± 7.3 | 72.9 ± 4.8 | 68.1 ± 17.6 | 78.3 ± 18.2 | 74.3 ± 10.4 | 71.7 ± 6.5 |

Potato plants were grown in the greenhouse in 2.5 litre pots.
Developing tubers were harvested in the spring after 10 weeks of growth and the amino acid contents were determined.
Data are presented in μmol gFW^{-1 and represent the mean ± SE of determinations on six individual plants per line.}

Example 2 Repeated analysis of transgenic ADK antisense potato plants under greenhouse conditions and in field trials. Transgenic lines AdK-20, -4, -2 and -24 were again grown under greenhouse conditions and in a field trial and were investigated and analysed for morphological and biochemical parameters.

When the transgenic plants were grown in the greenhouse side by with wild-type controls no phenotypic changes concerning either the aerial part of the plant or the tuber size, number or morphology, were observed in lines AdK-20 or AdK-4, however there were significant increases in the total tuber yield in lines AdK-2 and both tuber number and tuber yield in line AdK-24 (Table 5). Furthermore the specific density of the tubers was elevated in lines AdK-20, AdK-2 and AdK-24 indicating that these lines are most probably characterized by a higher starch content.

Table 5 Yield, tuber number, density and starch yield per plant of the antisense StpAdK plants. Potato plants were either grown in the greenhouse in 2L pots or under field conditions. In both cases determinations were carried out on fully senescent plants. Data represent the mean SE of determinations on six individual plants per line (greenhouse trial) or eight plants per line (field trial); values in bold type and underlined were determined by the t-test to be significantly different (P<0.05) from the wild type. n.m. = not measured.

| | | | | | |
|---|---|---|---|---|---|
| Parameter | WT | AdK-20 | AdK-4 | AdK-2 | AdK-24 |

Greenhouse trial 1999

| | | | | | |
|---|---|---|---|---|---|
| Total tuber yield (g) | 309 ± 37 | 287 ± 28 | 327 ± 41 | 366 ± 19 | 541 ± 57 |
| Tuber number | 8.3 ± 1.0 | 7.2 ± 1.4 | 10.4 ± 0.9 | 11.9 ± 2.1 | 16.6 ± 3.3 |
| Specific density | 1.087 ± 0.005 | 1.091 ± 0.007 | 1.093 ± 0.003 | 1.096 ± 0.002 | 1.099 ± 0.010 |

Field trial 2001

| | | | | | |
|---|---|---|---|---|---|
| Total tuber yield (g) | 867 ± 81 | 1460 ± 74 | 1486 ± 54 | n.m. | 1596 ± 97 |
| Tuber number | 7.9 ± 0.8 | 11.2 ± 0.3 | 10.6 ± 0.4 | n.m. | 7.9 ± 0.5 |
| Specific density | 1.072 ± 0.001 | 1.080 ± 0.003 | 1.081 ± 0.003 | n.m. | 1.081 ± 0.002 |
| Starch (g plant⁻¹⁾ | 106 ± 11 | 202 ± 10 | 209 ± 9 | n.m. | 224 ± 17 |

The total adenylate kinase activity was observed to be reduced down to 67% of that found in wild type plants in leaves and to a similar extent in tubers (Table 6).

Table 6. Enzyme activities in antisense StpAdK transgenic lines. Activities were determined in 6-week-old leaves or 10-week-old tubers. Data presented are mean SE of determinations on six individual plants per line (chloroplasts were only isolated from four plants per line). Values in bold type and underlined were determined by the t-test to be significantly different (P<0.05) from the wild type. n.m. = not measured, Sol. = soluble and GB=granule bound.Φ The activity of branching enzyme is expressed as fold-stimulation of glycogen phosphorylase.

| | | | | | |
|---|---|---|---|---|---|
| (μmol g FW^{-1min}⁻¹⁾ | WT | AdK-20 | AdK-4 | AdK-2 | AdK-24 |

Adenylate kinase activity

| | | | | | |
|---|---|---|---|---|---|
| Leaf | 21.3 ± 3.2 | 17.2 ± 1.4 | 17.4 ± 0.6 | 15.7 ± 2.2 | 14.4 ± 1.8 |
| Isolated chloroplast | 12.4 ± 1.7 | n.m. | 8.3 ± 12 | 7.1 ± 2.1 | 5.6 ± 0.9 |
| Tuber | 15.2 ± 2.1 | 12.0 ± 1.4 | 11.3 ± 0.9 | 10.6 ± 1.3 | 9.7 ± 1.7 |

Other tuber enzyme activities

| | | | | | |
|---|---|---|---|---|---|
| Phosphoglucomutase | 4117 ± 320 | 3941 ± 426 | 4307 ± 123 | 4021 ± 279 | 3871 ± 192 |
| AGPase | 519 ± 73 | 550 ± 34 | 637 ± 23 | 620 ± 61 | 717 ± 12 |
| Sol. Starch synthase | 143 ± 27 | 149 ± 7 | 108 ± 32 | 158 ± 32 | 109 ± 21 |
| GB Starch synthase | 31 ± 4 | 30 ± 3 | 27 ± 6 | 34 ± 4 | 31 ± 5 |
| Branching enzyme^{φ} | 6921 ± 1071 | 7324 ± 478 | 5824 ± 1291 | 7241 ± 2023 | 7841 ± 328 |

Furthermore, analysis of chloroplasts isolated from leaves of both wild-type and transgenic plants for adenylate kinase showed that this loss in activity was localized to the plastid which is in agreement with the sequence based deduced subcellular localization. There was little change in the activities of other enzymes of the starch biosynthetic pathway, with only the AGPase activity of line AdK-24 being significantly different (higher) than that found in the wild-type.

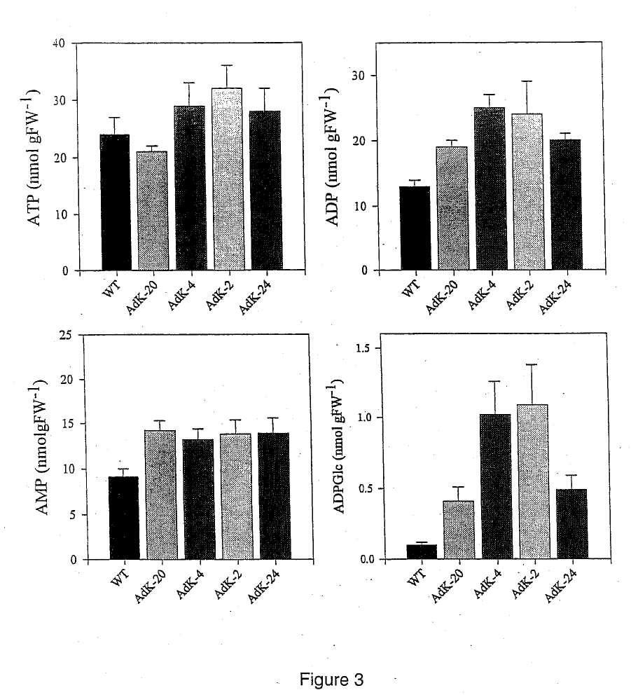

The reduction of StpAdK activity results in a general increase in the adenylate pools. As described above, adenylate kinase is catalyzing the interconversion of ATP, ADP and AMP. In order to analyze the effects of a reduced adenylate kinase activity on adenylate pools, the steady state levels of all three metabolites directly involved in the reaction and ADP-glucose were determined. The reduction in the activity of the plastidial adenylate kinase led to clear changes in the levels of the various adenylate pools (Fig. 3). Only in one of the most strongly inhibited lines, as expected on the basis of the increased pool size of individual amino acids, the total amino acid content of the tubers also exhibited a trendwise increase although due to the larger variation this increase was not statistically significant.

What is Claimed is:

1. A transgenic plant characterized in that the endogenous adenylate kinase (ADK) activity is reduced.

2. The transgenic plant of claim 1, wherein said ADK has a plastidial localization.

3. The transgenic plant of claim 1, wherein said ADK is encoded by a polynucleotide selected from the group consisting of:
(a) polynucleotides encoding a polypeptide having the amino acid sequence depicted in SEQ ID NO: 2;
(b) polynucleotides comprising the nucleotide sequence depicted in SEQ ID NO: 1;
(c) polynucleotides hybridizing to the complementary strand of the polynucleotide of (a) or (b); and
(d) polynucleotides the nucleotide sequence of which deviates from the nucleotide sequence of a polynucleotide of (c) due to the degeneracy of the genetic code.

4. The transgenic plant of any one of claims 1 to 3, wherein said endogenous ADK activity is reduced due to the presence of a foreign nucleic acid molecule in the genome of said plant.

5. The transgenic plant of claim 4, wherein said reduced endogenous ADK activity is achieved by an antisense, co-suppression, ribozyme or RNA interference effect or by in vivo mutagenesis, antibody expression or by the expression of a dominant-negative mutant.

6. The transgenic plant of claim 5, wherein said foreign nucleic acid molecule is complementary to the transcript of a gene encoding said ADK, operatively linked to a promoter allowing for expression in plants.

7. The transgenic plant of claim 6, wherein the promoter allows for constitutive expression in plant cells.

8. The transgenic plant of claim 1, which is a starch storing plant.

9. The transgenic plant of claim 8, which is a potato plant.

10. Transgenic plant cells, in which the endogenous ADK activity is reduced.

11. Propagation material or harvestable parts of a transgenic plant of claim 1 containing transgenic plant cells of claim 10.

12. A recombinant nucleic acid molecule comprising:
(a) a promoter ensuring transcription in plant cells; and operatively linked thereto
(b) a nucleotide sequence which, when transcribed in plant cells, leads to a reduction of the endogenous ADK activity in said plant cells; and optionally
(c) a transcription termination signal.

13. The recombinant nucleic acid molecule of claim 12, wherein the nucleic acid sequence defined in (b) is that of a nucleic acid molecule as defined in claim 1.

14. A process for producing transgenic plants displaying an increase in starch accumulation and/or in yield of starch storing parts, organs or tissues comprising the steps of:
(a) introducing into a plant cell a nucleic acid molecule the presence of which in the genome of said plant leads to a reduced endogenous ADK activity in plant cells;
(b) regenerating from transformed cells produced in step (a) plants; and optionally
(c) producing progeny from the transgenic plants produced in step (b).

15. Use of the recombinant nucleic acid molecule of claim 12 for the production of transgenic plants displaying an increase in starch accumulation and/or in yield of starch storing parts, organs or tissues.
